(19) United States
(12) Patent Application Publication
(10) Pub. No.: US 2016/0199581 A1
(43) Pub. Date: Jul. 14, 2016

(54) Needle Insertion Device

(71) Applicant: DEBIOTECH S.A., Lausanne (CH)

(72) Inventors: Astrid Cachemaille, Bussigny (CH); Laurent-Dominique Piveteau, Bussigny (CH); Paul Vescovo, Lausanne (CH)

(21) Appl. No.: 14/912,952

(22) PCT Filed: Sep. 11, 2014
(86) PCT No.: PCT/IB2014/064419

§ 371 Date: Feb. 19, 2016

(30) Foreign Application Priority Data
Sep. 11, 2013 (FR) FR1358744

(51) Int. Cl. A61M 5/31; A61M 5/50; A61M 5/32

(52) U.S. Cl. CPC A61M 5/31; A61M 5/32; A61M 5/5086; A61M 2005/3101

(57) ABSTRACT

The invention relates to a device and a method which allow force to be exerted on a compressible tissue in order to allow a needle to reach at least one of the layers of said tissue that could not have been reached by said needle without having compressed said tissue.

### FIELD OF THE INVENTION

The present document discloses a product and a needle insertion method in which the force and/or the depth of insertion and/or of injection can be controlled.

### PRIOR ART

To deliver a substance into dermal tissue with the aid of a needle, that is to say into an area extending a few millimeters below the level of the skin, there are two main methods:
The Mantoux method uses a “standard” needle of approximately 25-30 gauge mounted on a syringe. An example is given in FIG. 1. The needle is inserted at an angle of between 5° and 15° with respect to the surface of the skin, the bevel (the opening of the needle) being oriented in the direction of the surface of the skin. The angle is a crucial element to be maintained during the insertion. The needle in fact has a length greater than the thickness of the zone targeted during the injection. Too great an angle therefore risks an injection being performed in a tissue zone situated below the target zone, at too great a depth. The term “standard” is used here to distinguish needles that can be used in the Mantoux method from the other needles or microneedles used by the present invention.
The use of a needle having means for limiting the insertion of the needle or microneedle.

The Mantoux method is very disquieting and relatively painful since it requires a slow penetration of the needle, often accompanied by a forward and backward movement that is difficult to perform and requires considerable training. Only skilled practitioners are able to successfully perform this method. The main difficulty involves injecting to the correct depth and, in particular, in avoiding a situation where the injected liquid is located in the subcutaneous zone or escapes to the surface of the skin.

The other devices have a means for limiting the depth of insertion, either by the size of the microneedles or by the means that limit the insertion of the needle. These needles, generally called microneedles, have the advantage of limiting pain, of facilitating the work of the practitioner, and of mechanically limiting the depth of their insertion. However, on account of the shallow depth of insertion and the mechanical properties of the skin (elasticity, compressibility, etc.), these needles are often poorly inserted (incomplete insertion for example), which can lead to the injected substance leaking at the surface of the skin. Irrespective of the injection device using a needle or microneedle, a method may be needed to improve their insertion into the skin. This may involve a speed provided simply by a movement of the hand, or by a device allowing higher and better controlled speeds to be reached, or an aspiration or pulling of the skin. When the insertion is manual, the speed provided by the movement of the hand is often insufficient, as is shown, in FIG. 2a, by the insertion of a microneedle (1) with a length of approximately 700 micrometers. As the surface of the skin (2) is deformed, the penetration of the microneedle (1) into the dermal tissue (3) of the subject is only partial. Whether for hollow needles, coated needles, soluble needles or others, the penetration of the skin is insufficient and does not permit good assimilation of the substance to be administered within the tissue. In the case of hollow needles, there is in particular a high risk of the needle moving out of place and leaving the skin at the start of or during the injection, thus leading to a total loss (all of the fluid to be injected remaining at the surface of the skin) or partial loss (the start of the injection is carried out correctly and then, following withdrawal of the needle, the end of the injection does not take place normally and the liquid is thus lost). In the case of solid, coated or soluble needles, this means there is a risk of some of the medicament being present outside the tissue and of not being dissolved by the surrounding fluids. In other words, the depth of insertion is not optimal, and it is less than the length of the needle.

When the insertion is performed with the aid of an inserter (also called an insertion device), a microneedle has a greater likelihood of being correctly inserted, ideally completely inserted, into the dermal tissue. The inserter permits good control of the speed and force of the needle at the moment of penetrating the skin. FIG. 2b shows a microneedle (1) having penetrated dermal tissue (3) as a result of use of an inserter. In this example, the inserter allows the needle to reach a speed necessary for virtually complete insertion of the one or more needles. The depth of insertion reached here is equal to the length of the needle. The aim of this type of device is to pierce the biological barrier and allow the needle to correctly penetrate the target tissue in order to reach at most a layer of the tissue corresponding to the length of the needle, as shown in FIGS. 3a and 3b.

Preferably, the size of the needle thus determines the depth or location of the layer in which the substance has to be administered. The devices disclosed by the patent applications US 2011/0172639 and US 2012/0029434 comprise needles whose length determines the target layer. They are incorporated by reference into this application. In other words, the needles of these devices have a limited length, so as not to deliver the liquid at too great a depth. The length of the needle thus determines the layer to be reached.

The article “Echographic measurement of skin thickness in adults by high frequency ultrasound to assess the appropriate microneedle length for intradermal delivery of vaccines” (Laurent A., Mistretta F., Bottigioli D., Dahel K., Goujon C., Nicolas J. F., Hennino A. and Laurent P. E., Vaccine 25 (2007) 6423-6430), incorporated by reference into this document, concerned the thickness of the dermal tissue at four different sites of the human body: the thighs, waist, deltoid region and shoulder blades. According to this article, said thickness is variable depending on ethnicity, body mass index, injection site and gender. The study also concludes that the thickness of the skin (epidermis and dermis) varies between 1.66 mm and 2.77 mm. This article finally concludes that a needle with a length of 1.5 mm is universally suitable for at least the four sites studied.

Generally, after insertion of the needle, the delivery of the substance can be carried out (by an injection, the application of a substance, the dissolving of the needles, the application of a patch). However, as is shown in FIG. 2b, there is little or no space or cavity where the substance can be stored during and/or after the injection. The administration of the substance and its assimilation may be different depending on the characteristics of the dermal tissue. In FIG. 3a, the needle (1) is correctly inserted but, during the injection in FIG. 3b, the fluid resistance may cause a total or partial withdrawal of the needle and/or leaks (20), depending on the characteristics of the target tissue. The substance has no cavity in which to be stored during the injection, and therefore the substance spreads out of the tissue. This leakage phenomenon is all the more noticeable when the injection depth is shallow and/or a bolus is being injected (that is to say a substantial quantity of a fluid during a short injection period). In other words, the smaller the needle, the closer the injection will be made near the surface of the skin, which will increase the risk of leakage, and/or, the greater the quantity of the substance to be administered, the more the tissue will tend to push the substance back out from the skin during or after the injection and/or push the needle back, causing a leak. Another consequence of this absence of space a priori for the injected liquid is the need to quickly create a small pocket in which said liquid will be able to gather.

### GENERAL DESCRIPTION OF THE INVENTION

The present document discloses devices and methods permitting an improvement over the prior art.

Laurent A. et al. (2007) and the devices of the prior art use needles of a given length in order to inject and/or diffuse a substance, with the length determining a target layer. However, as is shown in FIGS. 2b and 3b, when the needle is correctly inserted, the skin completely envelops the needle, which induces a certain resistance to the injection and/or diffusion of the substance to be administered.

One of the principles of the invention is to exert a force on compressible tissue in order to allow a needle to reach one or more layers of said tissue that could not have been reached by said needle without having compressed said tissue. Preferably, these layers are deeper, that is to say farther from the surface of the skin, than the layers that would have been reached without this compression.

More generally, one of the principles of the invention is to control the stress exerted on, or the deformation of, the target tissue(s) (that is to say the one or more tissues in which the liquid is to be injected, or tissue against which a force is exerted by the device) before, during or after the insertion of the needle or before, during or after the injection, in order to
inject the liquid into tissue layers situated at a depth greater than the length of the needle, and/or
facilitate the injection of the liquid into the tissues (limited injection pressure, no leakage).

A first aspect of the invention is to permit the creation of a cavity and/or of a channel which is larger (for example longer) than the size of the needle, in order to create a sort of (virtual) reservoir and thereby allow the substance to be better assimilated by the tissue. In other words, one of the aims of the present invention is to facilitate the diffusion of a substance within the tissue by creating a cavity (also called a reservoir), which can have the form of a channel, in the area of the end of the needle. At the start of the injection, this reservoir will be able to easily accommodate the administered substance, thus initiating the creation of a larger volume at the end of the needle, capable of ultimately receiving the entirety of the injected volume. Even before the start of the injection, this cavity can additionally permit the creation of a supplementary exchange surface needed for the diffusion of the liquid injected into the tissue. The viscoelasticity of the tissue can permit enlargement of the volume and/or of the exchange surface of the cavity as the injection proceeds. Without this reservoir being present in the area of the end of the needle at the moment of the injection, the opening of the needle risks being obstructed by the tissue, for example. In this case, a very high injection pressure is then needed to push aside the tissue obstructing the opening of the needle, in order to deliver a fluid from the needle. In this configuration (that is to say when no cavity or channel has been created), the fluid then tends to invest the needle/tissue interface, and this is manifested by the start of a leak, which will become worse as the injection proceeds. This results in partial or zero injection of liquid. Another effect associated with this pressure is the risk of causing more pronounced pain during the injection.

Conversely, when a supplementary channel or cavity is created due to deeper insertion of the needle and relaxation of the pressure, all or some of the injected substance can be at least temporarily stored in said cavity, limiting the risk of leaks and/or of withdrawal of the needle during the injection and/or of pain when the substance is injected (due to the tearing of the tissue, which tearing is necessary for the creation of a space for receiving the injected solution). In fact, the skin is endowed with a degree of elasticity that allows it to deform and thus accommodate a small volume of liquid. Beyond a certain quantity, the deformation is no longer capable of absorbing a supplementary volume, and the skin tears locally (in most cases generating a painful sensation). Moreover, by creating this cavity, the administered substance comes into contact with a larger surface area of tissue, thereby potentially improving its assimilation.

A second aspect of the invention is to compress the tissues in order to allow a microneedle to reach deeper layers than those it could have reached without compression and solely on account of its length. In other words, one of the aims of the invention is to allow the pointed end of the needle to reach (temporarily or not) a depth (of the dermal tissue at rest, that is to say when unstressed) that is greater than the length of the needle. This therefore makes it possible to produce shorter needles than those normally used to reach a given layer. For example, by exerting a force on microneedles of 300 microns, these microneedles are able to reach a depth (of the dermal tissue at rest) which is normally reached only by microneedles measuring at least 1 mm. This therefore makes it possible to limit the length of the needles, which constitutes a real gain both as regards economy and also as regards the method of producing these needles.

A third aspect of the invention is to ensure the insertion of the needle to a given depth while limiting the speed of the needle when the latter comes into contact with the skin. The speed allows the needle to pierce the biological barrier and compensate for the deformation effect of the skin associated with its elasticity while the force exerted, and therefore the pressure, allows the needles to penetrate to a given depth. It is likewise possible to pull or pinch or aspirate the skin in order to pierce the biological barrier, then to exert a force on the needle in order to permit the insertion to a given depth. Thus, the insertion in this case takes place in two stages:
piercing the biological barrier of the tissue (for example the stratum corneum) with the aid of various means limiting the elasticity of the skin, such as speed, pulling, pinching or aspiration of the skin, or any other means known to a person skilled in the art,
driving the needle to a desired depth by compression of the tissues; the base of the needle or a surface substantially parallel to the tissue could permit compression of the target tissue.

A fourth aspect of the invention is to permit a sponge effect. In one possible embodiment, a compression means can exert a force on target tissue before, during or after the insertion. This compression can be effected by the base of the needle or by another element optionally independent of the base of the needle or of its support. The principle of this embodiment is to have a surface compressing a target tissue, then relaxing this force (suddenly or gradually) before, during or after the injection of the fluid.

This compression of the target tissue will allow the liquids present in this tissue to move toward “non-compressed zones”. The act of completely or partially removing this compression will create a sponge effect. In fact, the target tissue will tend to recover its original shape, thus creating an effect of suction of the displaced liquids but also of the fluid that is administered (application or injection of the substance, application of a patch, etc.). This sponge effect may be fairly long, and the assimilation of the administered substances after the removal of the needle can also be improved.

The sponge effect is also possible according to another embodiment which permits independent control of the pressure exerted by the base of the needle (P₁) (or needle support) and the pressure exerted by the distal end of the inserter (P₂) (preferably around the zone where the needle is positioned) on the target tissues. Preferably, for successful injection, these two pressures P₁ and P₂ must be different.

The pressure P₁ at the base of the needle must be sufficient to ensure that the liquid is delivered:
without leakage (the needle remains fully inserted throughout the injection),
possibly in layers which are situated at a depth greater than the length of the needle.

The pressure P₂ must be limited in order to allow the injected liquid to diffuse within the target tissues which are located at the periphery of the needle. An excessive pressure P₂, or one exerted too close to the injection point, or a combination of these two effects could result in a very high injection pressure, which would lead to leakage, as explained previously. In other words, in such an embodiment, the device must permit control of two distinct pressures in order to improve the injection into tissue or a target layer:
The pressure P₁ is generated independently of the user via a force F₁. This force F₁ can be created, for example, by an elastic element (examples: helical spring, pressurized gas), a mass subjected to gravity, the atmospheric pressure;
The pressure P₂ can be generated by the user or by a mechanism inherent to the device. In the case where the user generates the pressure P₂ via a force F₂, a visual system can indicate to the user that the bearing force F₂ is within the force range guaranteeing successful injection (FIG. 10). It is also possible to incorporate a safety mechanism which prevents the insertion of the needle when the force F₂ is not within the defined range, for example by prohibiting access to the trigger that releases the propulsion of the needle, or by using a trigger which is released by the mechanism once a minimum force F₂ is reached.

The pressure P₂ can be generated by a mechanism integral to the inserter via a force F₂.

To do this, the distal end of the inserter can comprise three independent parts:
the needle and its base subjected to a pressure P1 via a force F1,
a surface located around the injection zone and dedicated to controlling the pressure of the target tissues (pressure P₂ via a force F₂),
a surface located at the periphery of the inserter and dedicated to positioning and holding the latter in place on the tissues [FIG. 11]

The force F₂ can be created, for example, by an elastic element (examples: helical spring, pressurized gas), a mass subjected to gravity, the atmospheric pressure. The surface located at the periphery of the inserter and dedicated to positioning can be kept in contact with the skin by the user.

It is thus possible to generate a “sponge” effect, which allows the substance to be “aspirated” by the target tissues by controlling the pressure P₂ (for example by reducing said pressure). This reduction in pressure can allow the target tissues to recover their initial shape [FIG. 11]. It can take place during the injection or just before the injection in order to initiate or facilitate the diffusion within the target tissues. Since the tissues are viscoelastic, the return to the initial shape takes some time and can continue after the injection is completed. The sponge effect can thus be prolonged for a certain period of time. By virtue of this sponge effect, the assimilation of the administered substances can likewise be improved even after the removal of the needle.

The sponge effect can be increased if the pressure P₂ becomes lower than the atmospheric pressure. This can be achieved if the distal part of the inserter has a cavity to which a vacuum is applied. With this architecture, it is not essential that the inserter comprises a third part dedicated to positioning and holding in place on the skin. In fact, the peripheral part of the needle to which a vacuum is applied can be sufficient to position the device and hold it in place on the skin [FIG. 12]. It is possible to reverse the direction of the force F2 applied by the distal part of the inserter in order to increase the sponge effect [FIG. 11c]. A complete link between the distal part of the inserter and the adjacent tissues is then necessary.

In addition, the invention discloses a method comprising the following steps:
insertion of a needle,
application of at least one force:
   F₁ on said needle, which will generate a pressure P₁ on a target tissue in contact with the base of the needle: P₁=F₁/S₁, S₁ being the surface of the base of the needle in contact with the tissue; and/or
   F₂ at the distal part of the body of the inserter, which will generate a pressure P₂ on a target tissue P₂=F₂/S₂; S₂ being the surface at the distal part of the body of the inserter where the force F₂ is exerted (P₂ can be greater than, equal to or less than the atmospheric pressure),
optionally, reduction or cancellation of the pressures P₁ and/or P₂.

The application of the pressures P₁ and P₂ via the forces F₁ and F₂ can be effected before, during or after the partial or complete insertion of the needle.

By controlling one or both of the pressures, it is possible to reduce the injection pressure and leakages, create a sponge effect, reach one or more layers deeper than the length of the needle, distribute the substance over larger surface areas, and avoid and/or limit the effect of tearing of the tissue during the injection. With the pressure P₁, it is possible to reach one or more layers deeper than the length of the needle, create a channel or a cavity, and limit the impact effect during the insertion of the needle. The withdrawl or reduction of the pressure P₁ can permit a sponge effect. With the pressure P₂, it is possible to control the pressure in the target tissues, or even create a sponge effect, in order to easily deliver the liquid to the target tissues.

In the present document, the detailed description of the invention includes embodiments of devices, systems and methods that are presented by way of illustration. It will be appreciated that other embodiments are conceivable and may be applied without departing from the scope or spirit of the invention. Therefore, the detailed description given below must not be taken in a restrictive sense.

### LIST OF FIGURES

To allow a better understanding of the invention, one or more embodiments will be described which are shown in the figures attached to this document. Of course, the invention is not limited to these embodiments.

FIG. 1 is a photograph showing an intradermal injection by the Mantoux method.

FIGS. 2a and 2b are transverse sections showing the insertion of a microneedle with a length of 700 microns.

FIGS. 3a and 3b are schematic representations of the insertion of a microneedle and the injection of a substance when the tissue has not been compressed.

FIGS. 4a, 4b and 4c are schematic representations of the insertion of a microneedle with compression of the underlying tissue, and the relaxation of the tissue after compression of the latter.

FIGS. 5a, 5b, 5c and 5d are schematic representations showing different means for administering the substance into tissue that has been compressed.

FIGS. 6a to 6f are schematic representations showing the different types of needle that can be used by the invention (list not exhaustive).

FIG. 7 compares the depth of the channel generated upon insertion of a microneedle with or without compression of the tissue.

FIG. 8 is a schematic representation showing an embodiment of an inserter according to the principle of use of the invention.

FIGS. 9a, 9b and 9c partially disclose different embodiments.

FIGS. 10a and 10b are schematic representations showing two embodiments of an inserter with a safety system and a trigger indicator.

FIGS. 11a, 11b, 11c, 11d and 11e are schematic representations showing embodiments of an inserter with three distal parts.

FIGS. 12a and 12b are schematic representations of an inserter using negative pressure to increase the sponge effect.

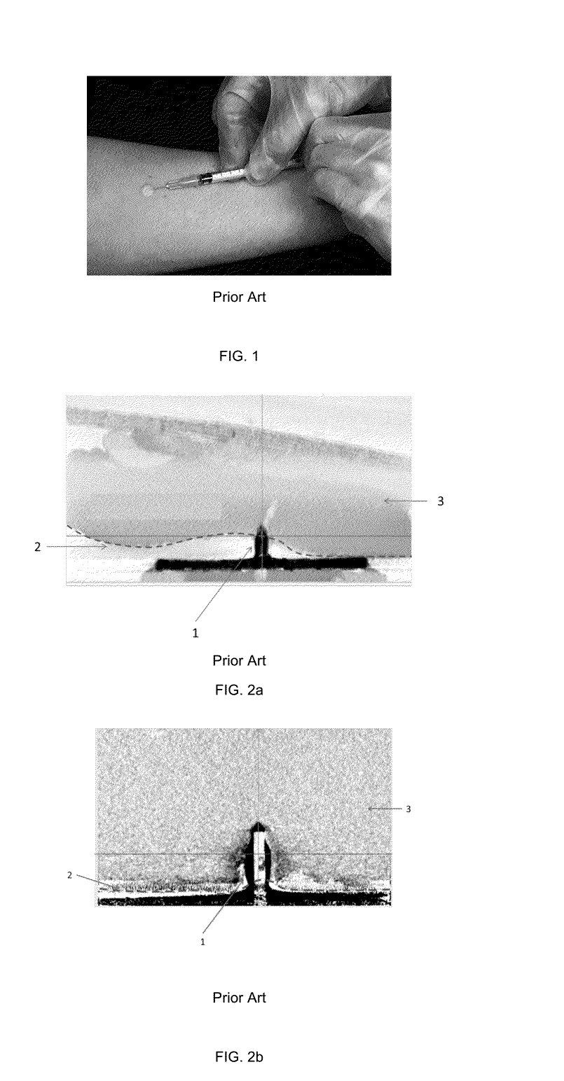

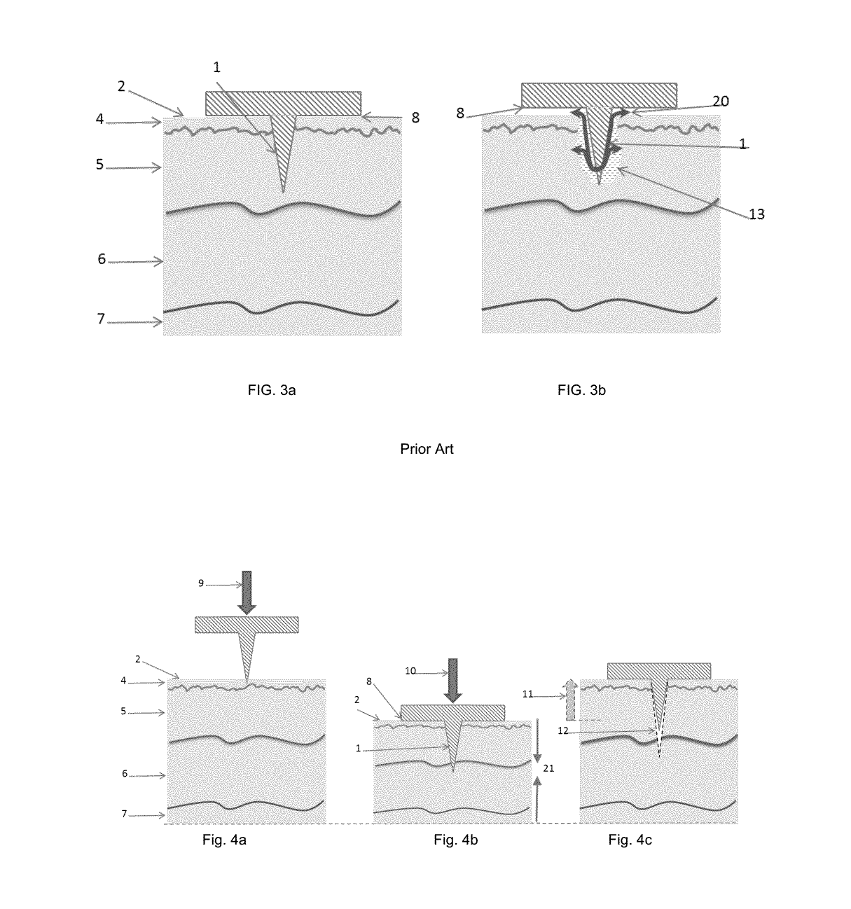

### LIST OF FEATURES

1 needle or microneedle
2 surface of the tissue
3 tissue
4 first layer of skin
5 second layer of skin
6 third layer of skin
7 fourth layer of skin
8 base of the needle
9 action A
10 action B
11 action C
12 channel or cavity
13 substance
14 patch
15 substance on the patch
16 opening
17 length of the needle
18 depth of insertion without tissue compression
19 depth of insertion with tissue compression
20 leakage
21 compression of the tissue
22 portion of the base of the needle extending parallel to the tissue
23 trigger
24 spring
25 needle support
26 body of the inserter
27 distal end
28 portion of the distal end of the inserter extending parallel to the tissue
29 face of the support intended to bear against the tissue
30 distal end of the needle
101 handle of the inserter
102 spring of the handle of the inserter
103 limit stop of the body of the inserter
104 skin formats a papule
105 force F₂
106 visual indicator
107 spring for generating the force F₂
108 handle for actuating the spring generating the force F₂
109 bearing surface for controlling the pressure of the target tissues
110 movable parts for controlling the pressure of the target tissues
111 adhesive positioned on the surface 109
112 orifice for applying a vacuum to the target tissues
113 chamber for applying a vacuum to the target tissues

### DETAILED DESCRIPTION OF THE INVENTION

The present invention is described and characterized by the independent claim(s), while the dependent claims describe other features of the invention.

Unless otherwise indicated, the scientific and technical terms used in the present document have meanings commonly used by a person skilled in the art. The definitions given in this document are mentioned in order to facilitate an understanding of the terms frequently used and are not intended to limit the scope of the invention.

The indications of direction used in the description and in the claims, such as “up”, “down”, “left”, “right”, “upper”, “lower”, and other directions or orientations are mentioned in order to afford greater clarity with reference to the figures. These indications are not intended to limit the scope of the invention.

The verbs “have”, “comprise”, “include” or equivalent are used in the present document in a broad sense and generally signify “includes, but without limitation thereto”.

In the present document:
A needle designated as “standard” is a needle used, for example, for the Mantoux method, that is to say a needle mounted directly on a syringe or connected to the latter without means for limiting the depth of insertion.
The terms “needle” and “microneedle” are used as equivalents.
The expression “a needle” is to be understood as “at least one needle”.
A hollow needle is a needle which allows a substance to be injected. Said substance passes through at least one channel of the needle and emerges through at least one opening. The opening can be beveled, on the side or at the tip.
A coated needle (or soluble needle) is a needle that comprises the substance to be administered over all or part of the needle or of its structure (for example as described by the international application WO2011/076537). The substance can likewise be stored in a cavity of the needle (for example when a needle comprises at least one cavity on the body and/or the base of the needle).
Other needle: needle which protrudes from a substrate and whose aim is to initially open the surface of the skin in order then to apply thereby, or by other means, a substance to the surface.

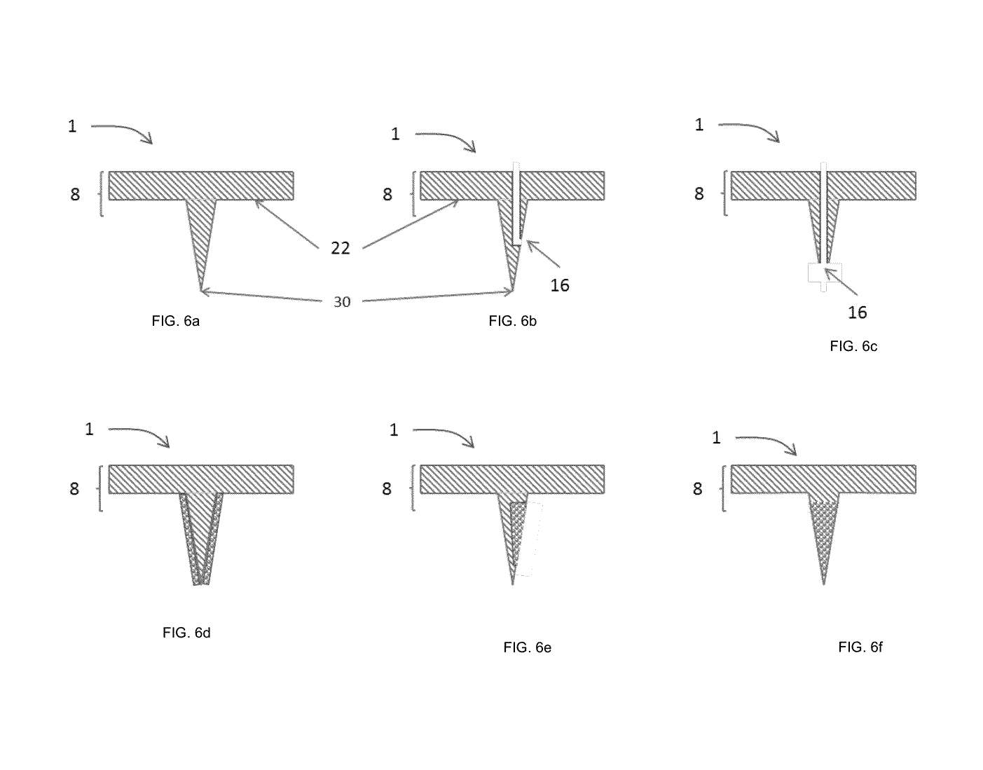

Different types of needles are represented schematically in FIGS. 6a to 6f. The needle (1) of FIG. 6a is a solid needle and therefore only permits perforation of the skin. The needles (1) of FIGS. 6b and 6c are hollow needles and thus comprise a channel and an opening (16) to allow a substance to be injected to a defined depth. Two examples are given here: in one case the opening is situated at the tip of the needle, while in the second case the opening is situated on the side of the needle barrel. It will be appreciated of course that other intermediate configurations or ones with multiple openings are conceivable, and also other needle geometries. The needles of FIGS. 6d to 6f are needles comprising a substance which is located directly on the needle and which forms all or part of the needle. All of these needles, and others, can be used according to the same principle of the invention. The needles (1) comprise a distal end (30) and a base (8) intended to come into contact with the surface of said tissue. Optionally, the base (8) can comprise a portion (22) extending parallel to the tissue and intended to come into contact with said tissue, this portion making it possible, for example, to stop the insertion of the needle and/or to compress the tissue.

The device used comprises a body defined by a proximal end and by a distal end which is intended to come into contact with tissue, and also a needle comprising a pointed distal end. The tissue to be penetrated is characterized by a greater or lesser compressibility. Said device is designed to permit control of the pressure applied to the tissue:
in contact with the base of the needle (characterized by a pressure P₁),
optionally, in contact with the distal end of the body of the inserter (characterized by a pressure P₂).

The device additionally comprises compression means for exerting one or more forces before, during or after the insertion of the needle, in order to compress target tissue.

In one embodiment, the compression means are neutralized suddenly or gradually after said pointed end of the needle has reached its target or a defined depth. The deactivation of the compression means can be effected manually or automatically.

In one embodiment, the device comprises a propulsion means designed to move said needle in the direction of said distal end of the device. Said propulsion means can be configured to reach a speed of between 1 meter per second and 100 meters per second. This speed may be reached only at the moment when the distal end of the needle comes into contact with the target tissue.

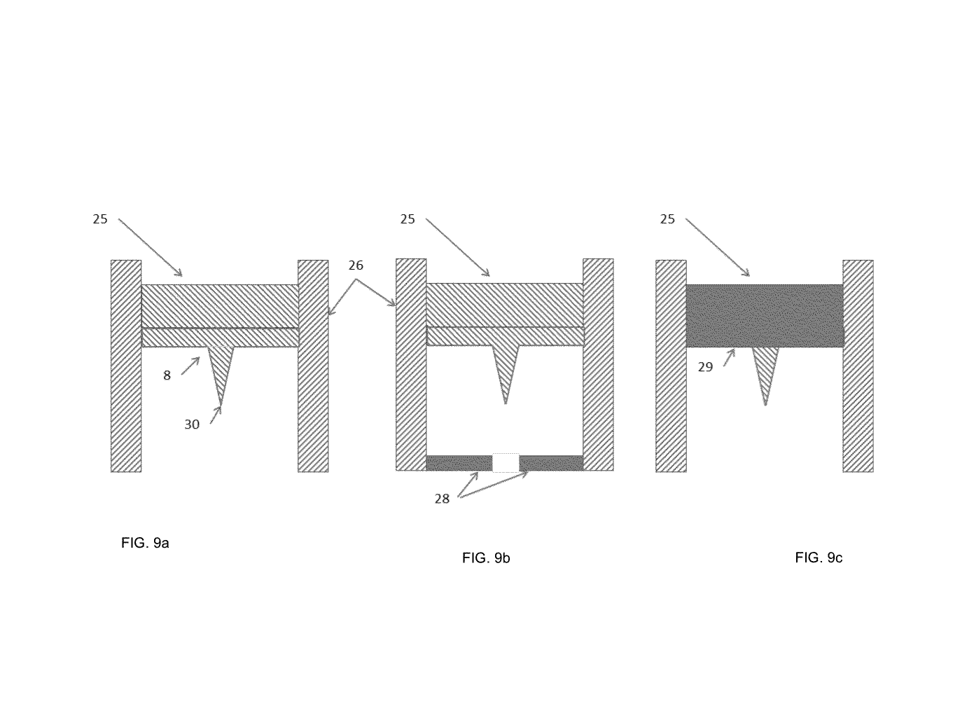

The sequential effect of the compression of the tissue will be understood from FIGS. 4a to 4c. In FIG. 4a, a needle (1) is inserted by virtue of an action A (9) making it possible to pierce the biological barrier of the tissue (2). This action A may be diverse in nature, for example pulling, pinching and/or aspirating the skin. In this example, it may be a propulsion means (24 as shown in FIG. 8) allowing the needle to reach a speed of between 1 m/s and 100 m/s at least before or when the distal end (distal with respect to the hand of the user, not with respect to the tissue) of the needle comes into contact with the tissue. This propulsion means (24) can be a spring, elastic and/or an elastic blade. In FIG. 4b, an action B (10) is applied in order to compress the tissue. In this example, by virtue of the base (8) which comprises a portion (22) parallel to the tissue, the target tissue is compressed and allows the needle to penetrate the tissue more deeply. Without this base, the needle would have potentially continued its insertion and it would not have been able to compress the tissue in order to reach a deeper layer. In other words, without a base or surface allowing the target tissue to be compressed, the depth of penetration is potentially equal to the length of the needle whereas, if a force is applied to the target tissue (if possible close to the insertion point, for example via the base of the needle), then the depth of penetration will be able to be greater than the length of the needle. In the present example, it is a portion of the base of the needle that exerts this action B against the tissue. However, it is possible that another element of the device exerts this action B on the tissue in order to have the same effect. In particular, this can be the needle support or an element independent of the needle. FIGS. 9a to 9c disclose different embodiments comprising a face intended to come into contact with the target tissue in order to compress the latter. In FIG. 9a, it is the base (8) or more precisely the portion (22) of the base (8); in FIG. 9b, it is a portion (28) of the distal end of the device, whereas, in FIG. 9c, it is a face (29) of the support. The compression of the tissue can be effected by various compression means such as a spring, an elastic blade, a piece of elastic and/or the user's hand exerting a force on the device.

The different layers (4, 5, 6, 7) or depth levels thus compress. In the present document, the layers (4, 5, 6, 7) represent different depth levels in order to better understand the effect of the compression of the tissue and the principle of the invention.

Optionally, but in a preferred manner, the action B can be totally or partially neutralized either suddenly or gradually. In FIG. 4c, the tissue, by virtue of its elasticity or its viscoelasticity, tends to return to its state of equilibrium exerting an action C (11).

Depending on the characteristics of the tissue, the channel or reservoir (12) created by the needle (1) materializes.

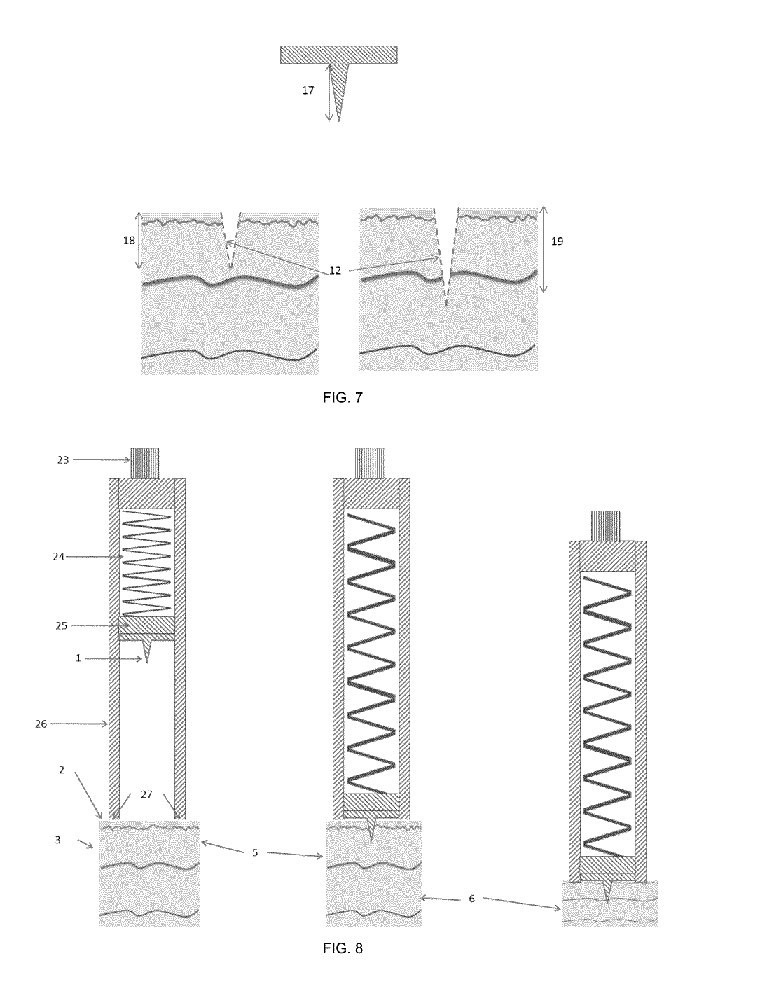

In FIG. 7 it is possible to compare the depths (18 and 19) of the channel (12). Since no action B has been applied to the tissue, in other words since the tissue has not been compressed during or after the insertion of the needle (1), the depth of insertion (18) corresponds at maximum to the length of the needle. When an action B is exerted on the tissue, as described in the present document, the depth (19) measured after the tissue has returned to its initial state is greater than the length of the needle.

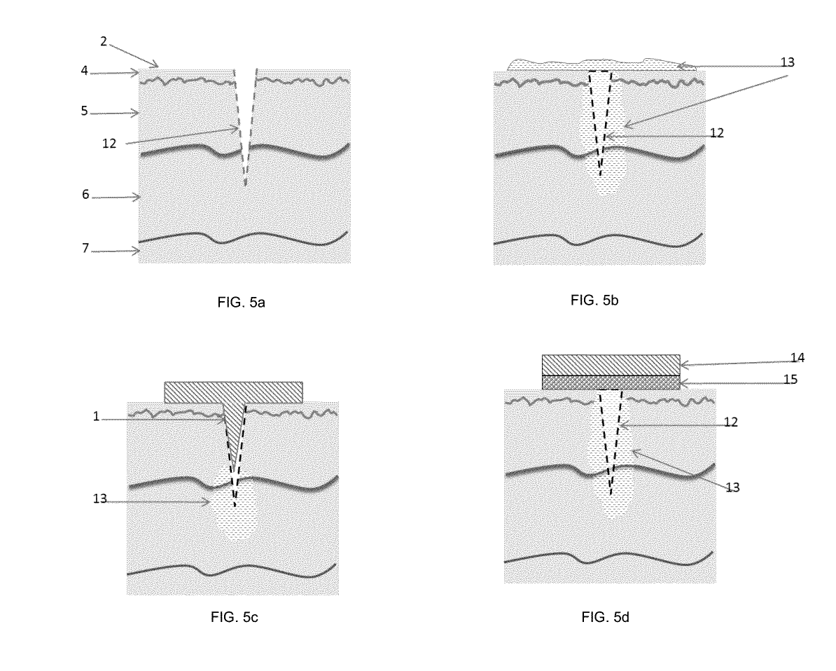

After insertion and compression of the tissue, the action B can be neutralized and the substance can be administered. In FIG. 5b, a pomade containing an active substance is applied to the cavity, which will allow said diffused active substance to easily pass through the protective layer of the skin and reach a given depth. In FIG. 5c, the needle diffuses its coating if it is a covered needle, dissolves in the case of a soluble needle or, if it is a hollow needle, an injection is carried out. In FIG. 5d, a patch containing the active substance is applied over the channel. In all of these configurations, with a return to the state of equilibrium, the cavity and the compressed tissue can have a sponge effect, which will improve the assimilation of the substance through the skin.

FIG. 8 is a schematic representation of a possible embodiment. The device comprises a needle (1) optionally attached (at least temporarily) to a support (25), said needle (1) comprises a distal end and is mounted movably inside the body (26) of the device. A propulsion means (24) allows said needle (1) to move in the direction of the distal end (27) of the device. In this example, the compression means exert a force in order to compress the target tissue and permit the total insertion of the needle into the tissue. By compressing the target tissue, the needle manages to reach the third layer (6), whereas the length of the needle only allows the second layer (5) to be reached. In this example, the compression means is the propulsion means (24).

### Control of the Force Applied:

The embodiments disclosed below may vary. The aim of such an embodiment is to ensure that the user applies a suitable force to the inserter at different moments of the application (from the insertion of the needle to the injection of the solution). It is therefore a system for controlling the force exerted against a target tissue via the base of the needle (comprising a surface substantially parallel to the target tissue) and/or via the distal end of the body of the inserter before, during and after the insertion and injection. In other words, by virtue of this control means, the user applies a defined force to a target tissue. By virtue of indicators (for example visual, tactile or acoustic), the user knows if he is applying the correct force at the correct moment. Indeed, by virtue of this embodiment, the user is able to apply a certain force to the target tissue and can then increase or reduce this force according to the requirements.

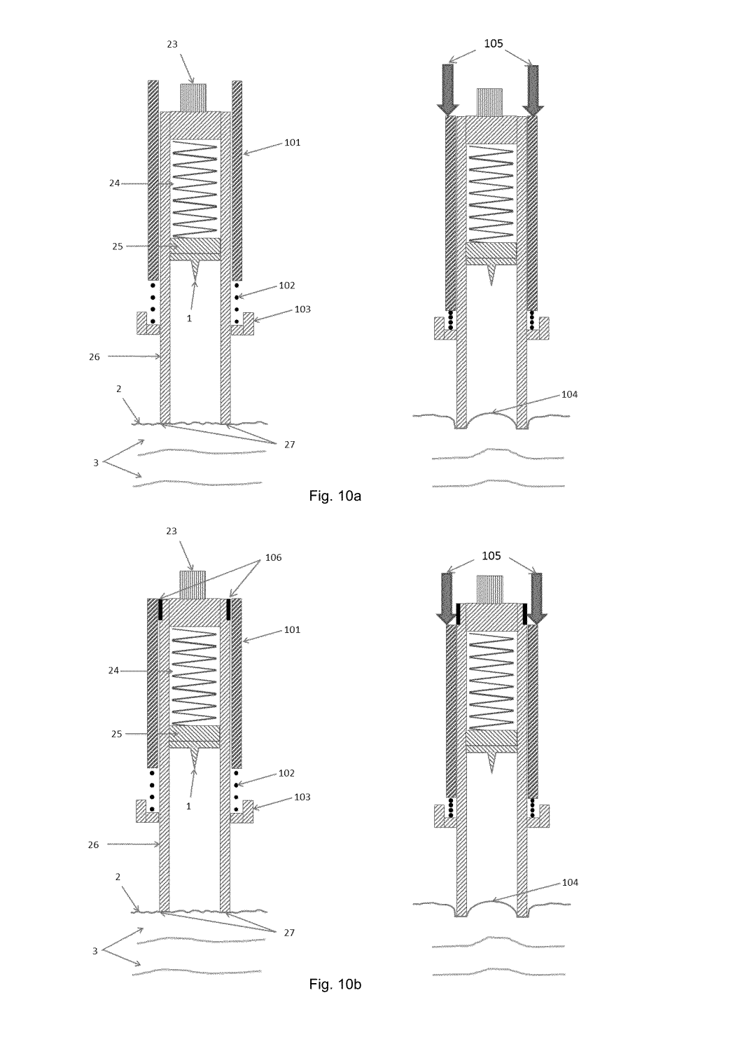

This control means can be arranged at the interface between the inserter and the user's hand (as disclosed in FIG. 10) or at the interface between the inserter and the target tissue (not disclosed by the figures). It can allow some triggering (triggering of insertion, injection, etc.) to be blocked as long as the force exerted is not within an acceptable force range. Or it can simply show the user some information (“force sufficient for insertion”, “force sufficient for injection”, etc.). It can also trigger the insertion automatically when a minimum force is reached. Thus, for example, the user applies a defined pressure on the target tissue during the insertion; optionally, the user can apply greater pressure in order to compress the target tissue and reach deeper layers. This new pressure can also be governed by the user by virtue of the control means. Before or during the administration of the solution, the user is able to control the pressure exerted in order to reduce the compression of the target tissues.

FIG. 10a illustrates an inserter equipped with a safety system which prevents the insertion of the needle when the pressure P₂ generated by the distal end of the inserter on the tissues (3) is not above a limit value P₂^{limit}. This guarantees optimal conditions for the insertion of the needle and/or the injection.

The safety system is composed of a handle (101), a spring (102) and a limit stop (103). The handle (101) slides on the body of the inserter (26). A spring (102) is inserted between the handle (101) and the limit stop (103) mounted on the body of the inserter. The spring (102) counters the movement of the handle (101) on the body of the inserter. When the user applies to the handle (101) an axial force F₂ (105) above a limit value F₂^{limit}, this compresses the spring (102) in such a way that the movement of the handle (101) renders the trigger (23) accessible. The triggering and thus the insertion of the needle are then possible. When the force F₂ is below the limit value F₂^{limit}, the movement of the handle (101) is not sufficient to permit triggering and, therefore, the insertion of the needle into the skin or the tissues. The spring (102) is dimensioned in such a way that the pressure P₂^{limit} corresponds to the force F₂^{limit}.

FIG. 10b illustrates an inserter equipped with a visual system (106) which shows the user that the pressure P₂ generated by the distal end of the inserter on the tissues (3) is above a limit value P₂^{limit}, which guarantees optimal conditions for the insertion of the needle and/or the injection. The visual system illustrated in FIG. 10b is very similar to the safety system described above and illustrated in FIG. 10a, except that the movement of the handle (101) does not render the trigger (23) accessible but instead uncovers a visual indicator (106) which shows the user that the bearing pressure P₂ of the inserter on the tissues is sufficient or adequate.

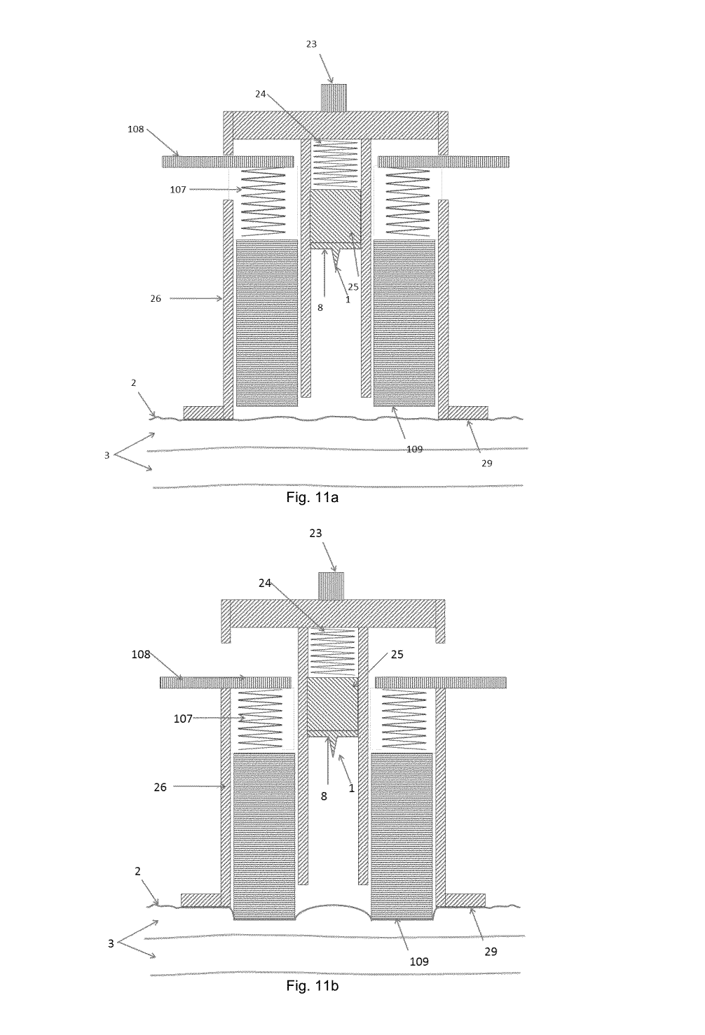

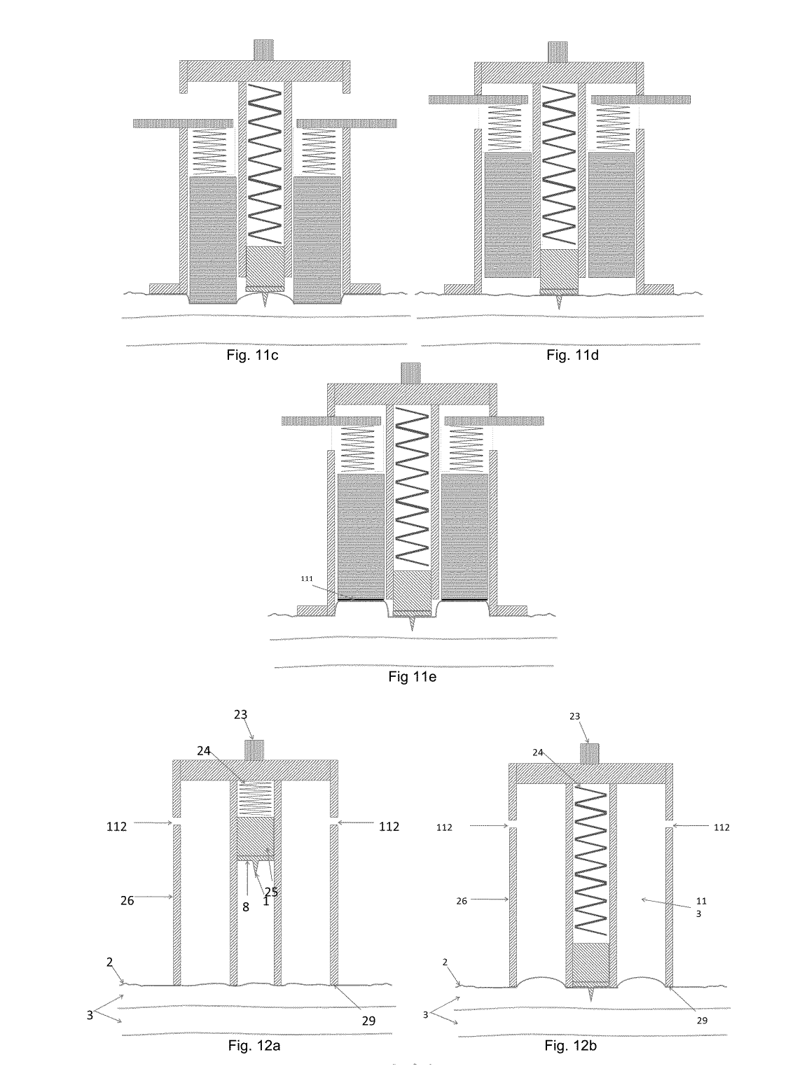

FIGS. 11a, 11b, 11c, 11d and 11e illustrate the functioning of an inserter with three distal parts. FIG. 11a illustrates an inserter comprising three distal parts and positioned on the skin via its peripheral bearing surface (29). The device additionally has a central distal bearing surface, formed by the base of the needle (8), and an intermediate distal bearing surface (109), which is arranged around the injection zone and is dedicated to controlling the pressure on the peripheral target tissues. Once the inserter is positioned on the tissues, the handles (108) are moved in order to move the mobile parts (110) in the direction of the tissues. When the handles are in the bottom position, the target tissues are subjected to the pressure P₂ generated by the springs (107) via the surfaces (109) (FIG. 11b). The insertion of the needle can then be effected (FIG. 11c). The handles are then moved away from the tissues (FIG. 11d), the effect of which is to cancel the pressure P₂ and generate a sponge effect on the target tissues, which then tend to recover their initial shape and aspirate the injected liquid.

The effect can be increased by positioning or activating an adhesive (111) on the surface (109) before use of the inserter. When the handles (108) are moved closer to the tissues, the surface (109), by virtue of the adhesive, connects completely to the target tissues. After insertion, when the handles (108) are moved away from the distal part of the inserter, the tissues then remain completely connected to the surfaces (109), which tends to increase the sponge effect described above (FIG. 11e).

FIGS. 12a and 12b illustrate an inserter using a vacuum to increase the sponge effect of the target tissues. The inserter is positioned on the tissues (FIG. 12a). The needle is inserted into the tissues, and a vacuum is then applied in the chamber (113) via the orifices (112). Applying a vacuum to the target tissues will tend to aspirate the injected liquid.

The vacuum can be applied in two stages. A first vacuum that is applied serves to position and maintain the inserter in place on the tissues, after which the insertion of the needle can be performed. The vacuum level can then be increased in order to generate the sponge effect. The intradermal injection of liquids having a viscosity greater than that of water presents some difficulties. In this context, applying a vacuum to the target tissues is of particular interest. This is because it can allow viscous liquid to be injected, which is otherwise too difficult, if not impossible.

It is understood here that the means for controlling the force applied to target tissue is not necessarily linked to an inserter having compression means for compressing the tissue in order to reach a layer deeper than the length of the needle. In other words, such a device may simply comprise:
a body comprising a proximal end and a distal end, the latter intended to come into contact with target tissue,
a needle mounted on a support that is movable inside the body of the inserter,
propulsion means intended to move the support and its needle in the direction of an insertion site on the target tissue,
pressure control means intended to allow a user to apply a defined force to the target tissue (compression or release of pressure).

These pressure control means can take different forms. They can comprise elements that slide along an axis of the inserter, at least one limit stop intended to limit the movement of this sliding element, counterforce means (an elastic blade, a spring, etc.) preferably exerting a force along an axis perpendicular to the surface of the target tissue, an indicator or a locking mechanism prohibiting insertion or injection when the force exerted by the user is not within a predefined range.

These pressure control means can be arranged either at the distal end of the inserter or at a zone where the user is intended to grip the inserter during use.

According to one embodiment, the device comprises a body defined by a distal end, intended to come into contact with said tissue, and a proximal end, at least one hollow needle of a given length intended to administer a solution, having a distal end and being mounted movably inside said body, a propulsion means designed to move said needle in the direction of the distal end of said body. Preferably, the device additionally comprises a compression means designed to temporarily compress said tissue at least in or near the insertion zone, in such a way that said pointed end of the needle, having penetrated the tissue, reaches a greater depth than the insertion of the needle without compression of said tissue. The compression means can be designed so as to no longer compress the tissue at least before the end of the administration of the solution.

The compression means can compress the target tissue for a defined duration. The compression means can be designed to compress said tissue during the insertion of said needle into the tissue. The compression means can be designed to compress said tissue after the distal end of the needle has penetrated the tissue. The compression means can be designed to no longer compress said tissue when said distal end of the needle has reached a defined depth. The compression means can be automatically or manually deactivated. Said needle can comprise a base intended to come into contact with the surface of said tissue once the needle has been inserted into the tissue. Said base can comprise a portion parallel to the surface of said tissue, intended to come into contact with said tissue. The compression means can exert a force against said base in order to compress said tissue. The compression means can also be the propulsion means. The length of said needle can be less than 3 mm. The depth of the channel resulting from the insertion with compression can be greater than or equal to 1.10 times the length of said needle. The needle can be driven at a speed in the range from 1 meter per second to 100 meters per second when said needle comes into contact with said tissue. The compression means can exert a force of 0 N to 200 N against said tissue during and/or after the insertion of said needle. The compression means can be a spring, elastic, an elastic blade, a pneumatic system, a hydraulic system or an electronic system. The needle can be fixed to a support against which the propulsion means exerts a force, at least during the driving of said needle toward said distal end. The needle can be fixed to a support designed to come into contact with said tissue in order to compress the latter in cooperation with the compression means. The needles (or at least one) can be hollow needles, coated needles or soluble needles. The device can additionally comprise an administering or sampling means designed to administer a substance into the tissue or to collect a sample. The compression means can be deactivated gradually or suddenly before or during the administration of a substance. The device can free the space needed for the formation of a papule during the administration of a substance into the tissue. The device can comprise a means for controlling the pressure exerted on the inserter by the user. The device can comprise a safety mechanism intended to authorize or trigger the insertion of the needle or the administration of the solution only when the pressure exerted on the target tissue is within a defined range. The device can comprise an indicator intended to inform the user that the pressure exerted on the target tissue is admissible for the insertion of the needle and/or the administration of the solution. The device can comprise a pressure applicator intended to generate negative or positive pressures on said target tissue or on a surface peripheral to said target tissue. The pressure applicator can compress or exerts a positive force against said target tissue or on a surface peripheral to said target tissue before, during and/or after the insertion of the needle. In other words, the pressure applicator can compress the target tissue, for example, with a force that is defined in accordance with the target tissue. The pressure applicator can exert a negative force or aspirates said target tissue or on a surface peripheral to said target tissue before, during and/or after the administration of the solution. In other words, the applicator can generate a lower pressure in the target tissue for example. This negative pressure is a pressure relative to the atmospheric pressure. It can entail an aspiration of the target tissue or entail pulling the target tissue in the direction of the proximal end of the device. The pressure applicator can exert a negative force or aspirates said target tissue or on a surface peripheral to said target tissue before, during and/or after the insertion of the needle.

According to another embodiment, the device comprises a body defined by a distal end, intended to come into contact with said tissue, and a proximal end, at least one hollow needle of a given length intended to administer a solution, having a distal end and being mounted movably inside said body, a propulsion means designed to move said needle in the direction of the distal end of said body. The device can additionally comprise a pressure applicator intended to generate negative or positive pressures on tissue. The pressure applicator can exert a first force on said target tissue before, during and/or after the insertion of the needle, then a second force on said tissue before, during and/or after the administration of the solution. Preferably, the first force and the second force can be different in terms of absolute value, relative value or the sense in which the force is exerted (for example in the direction of the proximal end or in the sense of the insertion of the needle). The first force can be positive or compels the target tissue to compress. The second force can be negative or aspirates or pulls the target tissue in the direction of the proximal end of the inserter, in which case the first force may be less than the second force.

The invention likewise discloses a method by which a needle, for example a hollow needle, is inserted into compressible tissue, which method can comprise the following steps (preferably in succession):
procuring at least one needle of a given length comprising a distal end, intended to penetrate the tissue, and a proximal end, designed not to penetrate the tissue,
inserting said needle into said tissue,
applying a force to said tissue
   in order to compress said tissue in such a way that said distal end, having penetrated the tissue, creates a channel that is longer than the channel resulting from the insertion of the needle without compression of said tissue, and/or
   in order to promote a sponge effect in the target tissue or neighboring tissue, and/or
   to aspirate or pull the target tissue.

The method can additionally comprise the following step: gradually or suddenly withdrawing said force before or during the administration of a substance. The method can additionally comprise the following step: withdrawing said needle. The method can additionally comprise the following step: administering a substance to said perforated tissue with the aid of a patch by applying a fluid to said tissue.

1-37. (canceled)

38. A device for intradermal injection of a solution, the device comprising:
a hollow needle for insertion into intradermal tissue and injecting the solution into the intradermal tissue;
a body defined by a distal end and a proximal end and forming a main axis, the distal end configured to come into contact with a surface of the intradermal tissue; and
a pressure applicator configured to generate a negative or a positive pressure on the intradermal tissue,
wherein the pressure applicator exerts a first force before the injecting of the solution, and exerts a second force during or after the injecting of the solution, and
wherein the pressure applicator is configured to set the first force and the second force based on a viscoelasticity of the intradermal tissue so as to prevent leakage of the solution to the surface of the intradermal tissue.

39. The device as claimed in claim 38, wherein the first force and the second force are exerted by the pressure applicator in a direction defined by the main axis and in opposite senses.

40. The device as claimed in claim 38, wherein the first force exerts a positive pressure on the intradermal tissue or compels the intradermal tissue to compress.

41. The device as claimed in claim 38, wherein the second force exerts a negative pressure on the intradermal tissue or aspirates or pulls the intradermal tissue.

42. The device as claimed in claim 38, wherein the hollow needle is mounted movably inside the body.

43. The device as claimed in claim 42, wherein at least one of the hollow needle and a needle support includes a base having an adhesive for affixing to the surface of the intradermal tissue.

44. The device as claimed in claim 43, wherein during or after the injecting of the solution, the pressure applicator is configured to move the hollow needle towards the proximal end of the body to pull the intradermal tissue.

45. The device as claimed in claim 38, wherein the pressure applicator is configured to generate a pressure lower than an atmospheric pressure inside the body.

46. The device as claimed in claim 38, further comprising:
a control device permitting a user to set at least one of the first force and the second force.

47. The device as claimed in claim 46, wherein the control device includes a visual indicator to indicate when at least one of the first force and the second force have not been reached.

48. The device as claimed in claim 46, wherein the control device includes a system preventing at least one of the insertion of the hollow needle and the injecting of the solution when one of the two forces has not been reached.

49. A method for intradermal administration of a solution by a device, the device having a hollow needle configured to inject the solution into intradermal tissue and body defined by a distal end and a proximal end forming a main axis, the method comprising the steps of:
applying a first force to the intradermal tissue before the injection;
injecting the solution into the intradermal tissue; and
applying a second force to the intradermal tissue during the step of injecting or after the step of injecting,
wherein the device is configured to set the first force and the second force based on a viscoelasticity of the intradermal tissue so as to prevent leakage of the solution to the surface of the intradermal tissue.

50. The method as claimed in claim 49, wherein the step of applying the first force and the step of applying the second force are exerted in a direction defined by the main axis of the body and in opposite senses.

51. The method as claimed in claim 49, wherein the step of applying the first force exerts a positive pressure on the intradermal tissue or compels the intradermal tissue to compress.

52. The method as claimed in claim 49, wherein the step of applying the second force exerts a negative pressure on the intradermal tissue or aspirates or pulls the intradermal tissue.

53. The method as claimed in claim 49, further comprising the step of:
indicating by a visual indicator whether at least one of the first force and the second force has not been reached.

54. The method as claimed in claim 49, further comprising the step of:
preventing the step of injecting the solution or a step of inserting the hollow needle into the intradermal tissue when at least one of the first force and the second force has not been reached.
